(19) United States
(12) Patent Application Publication
(10) Pub. No.: US 2010/0179397 A1
(43) Pub. Date: Jul. 15, 2010

(54) Device And Method For Continuous Monitoring Of A Chemical Parameter Of An Individual

(76) Inventors: Frank V. Bright, Williamsville, NY (US); Jerome J. Schentag, Amherst, NY (US); David T. D'Andrea, Getzville, NY (US)

Correspondence Address: HODGSON RUSS LLP;THE GUARANTY BUILDING, 140 PEARL STREET, SUITE 100, BUFFALO, NY, 14202-4040, US

(21) Appl. No.: 12/685,480

(22) Filed: Jan. 11, 2010

Related U.S. Application Data
(60) Provisional application No. 61/143,514, filed on Jan. 9, 2009.

(51) Int. Cl. A61B 5/00

(52) U.S. Cl. 600/309

(57) ABSTRACT

A device according to an embodiment of the invention includes a microdialysis catheter having an inlet and an outlet, a pump, and a flow-through monitor. The monitor is configured to analyze dialysate present in perfusion fluid exiting the microdialysis catheter. A chemical parameter may thereby be continuously monitored.

### CROSS-REFERENCE TO RELATED APPLICATION

This application claims the benefit of priority to U.S. provisional patent application Ser. No. 61/143,514, filed on Jan. 9, 2009, now pending, the disclosure of which is incorporated herein by reference.

### BACKGROUND OF THE INVENTION

In recent years, “point of care” laboratory technology has been described wherein a device, containing a means of body fluid collection, a measurement method, and a transmitter, has been seen by the medical profession as a possible means to monitor various body environments during care of patients. It has been suggested that such point of care devices could be implanted, ingested, or otherwise placed at desirable locations in the body and could be engineered to transmit critical data to a remote receiver located outside the body. Current continuous monitoring devices are extremely rare on the market, primarily because the sensors involve biological methods and these are not useful for continuous monitoring. In addition, most current devices are limited to sensing of physical parameters, and not chemical (biological) parameters.

### BRIEF SUMMARY OF THE INVENTION

The present disclosure provides an improved point of care laboratory that is arranged to continuously sense one or more chemical parameters within a mammalian body, and to transmit such parameters to an extra-corporeal receiver. The present disclosure incorporates the capabilities of many different laboratory instruments into one testing system that can be attached to the body of an individual (human or animal).

Chemical sensing (including biological sensing—e.g., sensing proteins) adds a new dimension to the capabilities of this type of device. New diseases are added to the list for diagnosis and monitoring by physicians. Among them include bleeding, cancer, diabetes, infections, inflammatory diseases in tissues and wounds, and possibly others. Current biological sensor methods do not work in the modern hospital because they only measure once at one location. Current systems also require an instrument and a new reagent cartridge for every measurement of a target analyte. The device and method here disclosed allow continual measurements for at least 72 hours, without change of sensor element. This is an improvement in the capability of multi-analyte detection for patient care.

### DESCRIPTION OF THE DRAWINGS

For a fuller understanding of the nature and objects of the invention, reference should be made to the following detailed description taken in conjunction with the accompanying drawings, in which:

FIG. 1 is a schematic of an embodiment of the presently disclosed system and method;

FIG. 2 is a schematic of a cassette for a wearable laboratory system according to another embodiment of the presently disclosed system and method;

FIG. 3 is a wearable laboratory according to another embodiment of the present disclosure shown attached to the arm of an individual and communication with a receiver;

FIG. 4 is a schematic of a continuous monitoring flow cell according to another embodiment of the present disclosure;

FIG. 5 depicts new and improved sensing modalities—Site Selectively Templated and Tagged Xerogels (SSTTX) and Protein Imprinted Xerogels with Integrated Emission Sites (PIXIES);

FIG. 6 is a graph of the fluorescent intensity of glucose at different concentrations in a flow cell over time;

FIG. 7 is a schematic of a microneedles device according to another embodiment of the present disclosure;

FIG. 8 is a schematic of a microneedles device according to another embodiment of the present disclosure; and

FIG. 9 is a schematic of a microneedles device according to another embodiment of the present disclosure.

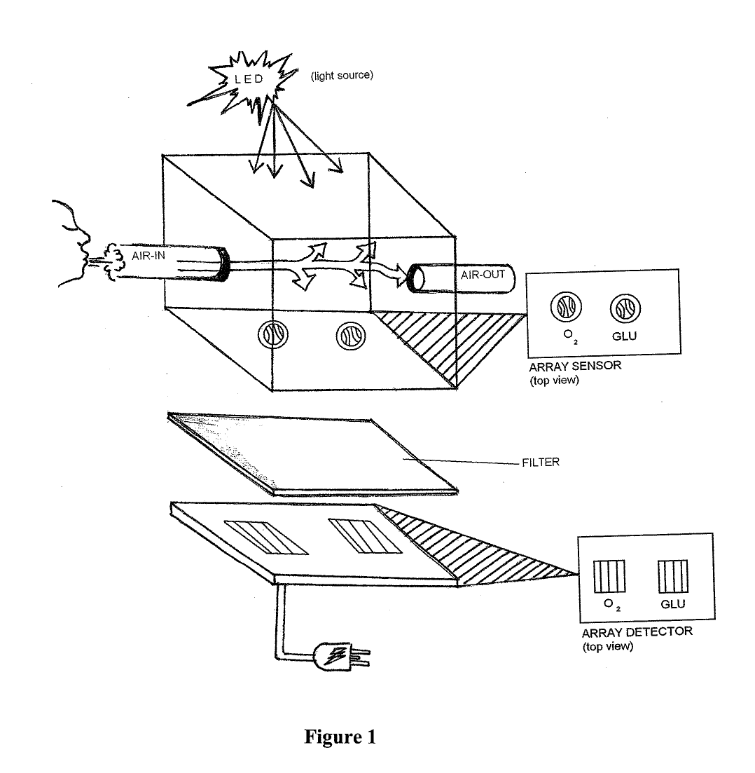

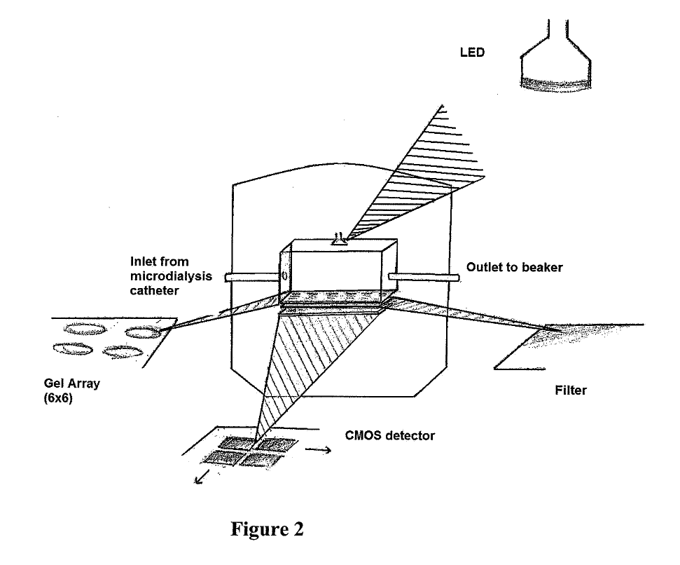

### DETAILED DESCRIPTION OF THE INVENTION

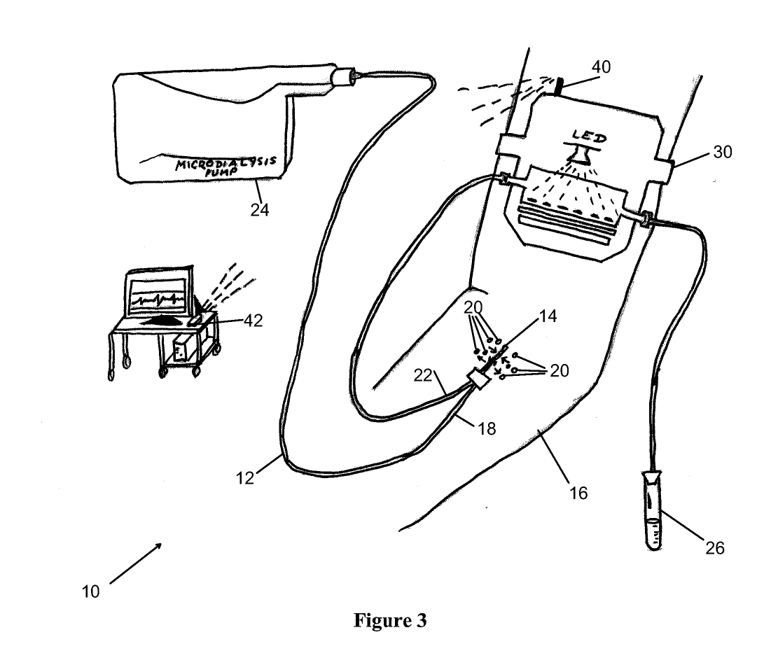

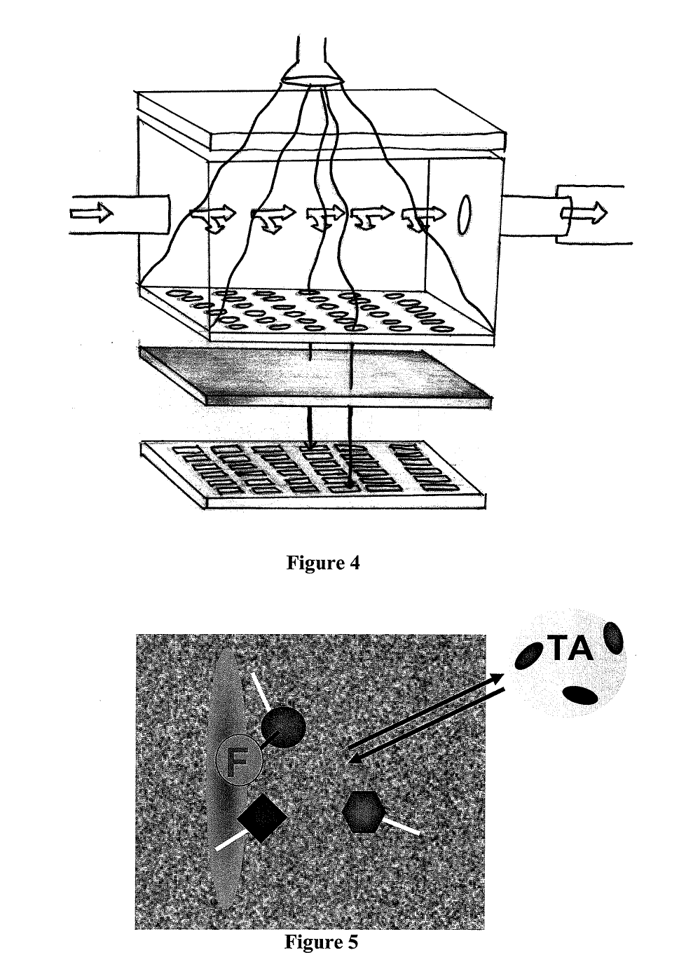

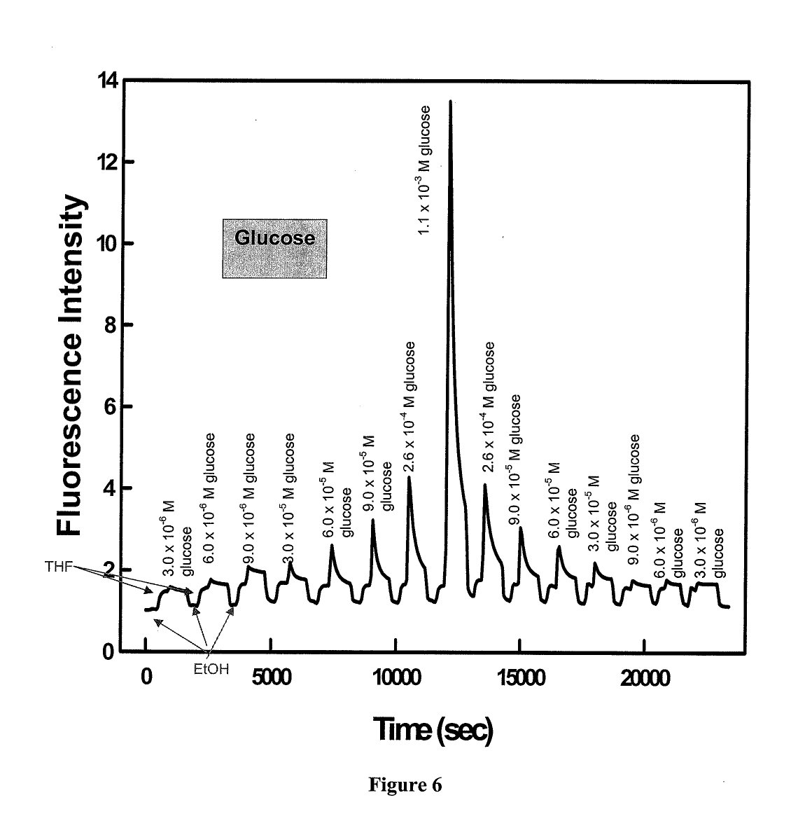

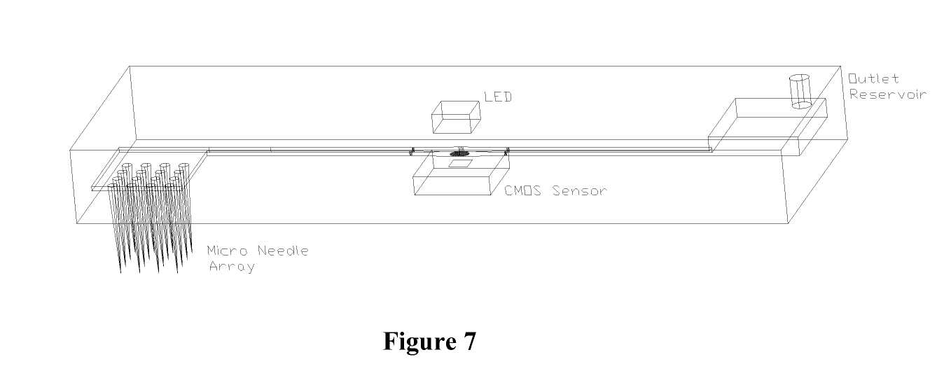

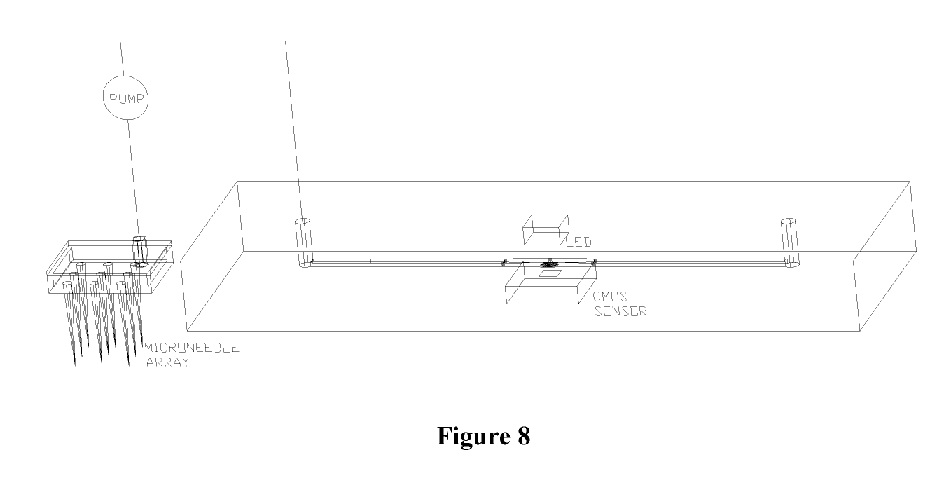

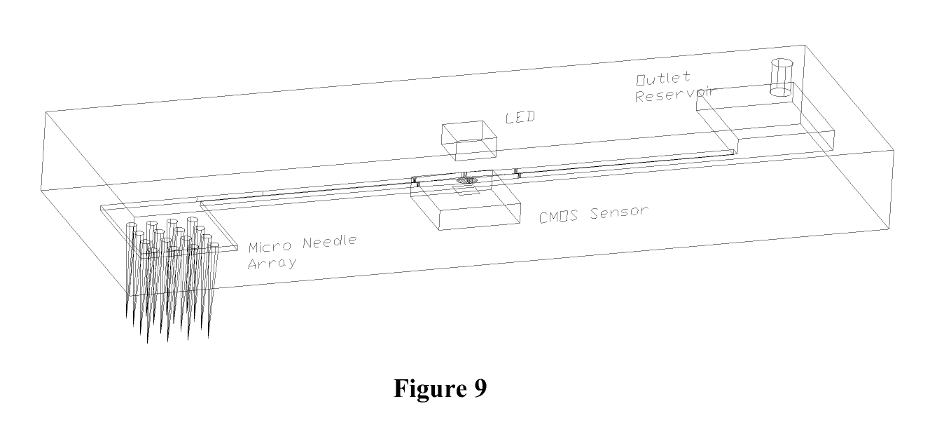

FIG. 3 depicts a device 10 according to an embodiment according to the present disclosure includes a microdialysis catheter 12. The microdialysis catheter 12 is configured such that a permeable portion 14 may be inserted into an individual 16. The microdialysis catheter 12 has an inlet 18 for receiving a perfusion fluid. As perfusion fluid flows through the permeable portion 14 of the microdialysis catheter 12, dialysate 20 may flow through the permeable portion 14 and exit the microdialysis catheter 12 by way of an outlet 22. The device 10 also includes a pump 24 in fluid communication with the inlet 18 of the catheter 12. The pump 24 is configured to cause perfusion fluid to through the microdialysis catheter 12. The device 10 further includes a flow-though monitor 30 in fluid communication with the outlet 22 of the catheter 12. The monitor 30 is configured to analyze the dialysate 20 present in the perfusion fluid, and, thereby, detect a chemical parameter of the individual 16. The monitor 30 may be removably attached to the individual 16.

The device 10 may further comprise a reservoir 26 in fluid communication with the monitor 30. The device 10 may further comprise a transmitter 40 configured to transmit monitored data from the monitor 30 to a receiver 42.

The present invention may be embodied in a method for continuously monitoring a chemical parameter of an individual. A device as described above is provided, and perfusion fluid is pumped through the microdialysis catheter, from the inlet, through the permeable portion, and exiting the outlet. The perfusion fluid, now containing dialysate is caused to flow through the monitor. The flow-through monitor is used to analyze the dialysate present in the perfusion fluid and to detect the chemical parameter.

A unique capability of the presently disclosed system and method is continuous monitoring of a number of different substances from the body of a patient. The point of care device will enhance the goal of real-time disease detection and would help with treatment during times when immediate information is essential. When configured into a minimally invasive microfluidics based monitoring chip, the sensor platform will bring real time continuous monitoring of vital functions to the care of patients that may need such monitoring, as with ICU patients. All of these features expand the market of both point of care laboratory testing and ICU monitoring, and in most cases the disclosed system and method will enhance the existing laboratory monitoring systems with improved accuracy and precision. In some aspects a point of care device is seen as one that is small enough to be easily used at bedside, and can be biologically inert, disposable and inexpensive. The transmission signal is sufficiently strong to be received by a remote receiver, which may be located apart from the patient's body so that the patient would have freedom of movement, or, be small enough to be carried by the patient. The outer shell of the system can be made from a polycarbonate or other suitable substance.

In some aspects the disclosed system and method has a flow cell for continuous sensing that may have a reagentless design. The fluid path can be by microdialysis or microneedles. One embodiment of the disclosed system and method has a laboratory sample sent on an “event trigger.” In this manner, an event trigger is used to trigger further sampling, thereby allowing medical treatment to become more proactive. The presently disclosed system and method is also useful for “signal averaging/AUC” like measurement in unstable patients.

Possible analytes that can be monitored continuously by the presently disclosed system and method include: BUN, creatinine, bilirubin/liver enzymes, lipids, electrolytes, glucose/Free Fatty Acids, pH, oxygen, lactate, vancomycin, antibiotics, sedatives, anti-sepsis drugs, cytokines (IL6, IL1, TNF, etc), C-Reactive Protein/procalcitonin, proteins/peptides, troponin, and various other chemicals and drugs. This list should be considered representative and not exhaustive. Monitoring all of the above continuously with currently available technology would necessitate two or three different detection instruments. However, by using Xerogel and fluoresence detection, one detection and one analysis system may be used for a very wide array of the analytes that are currently monitored in ICU patients.

Microdialysis is an optional method of analysis for most molecules; however, in some cases, samples may have to be sent to a laboratory, or a bedside laboratory instrument can be implemented. Thus, one would have a means of continuously monitoring the fluid output. Continuous monitoring can be used as a trigger for spot sampling, such as in ID, transplant rejection, CV collapse (sepsis), etc.

The disclosed system is a “reagentless” sensor, capable of continuous monitoring, and bedside display of the results of that monitoring. It can enable the common detection of all analytes, it is inexpensive to manufacture, the use of microdialysis avoids the drawing of blood, it can lead to less biofouling (longer life-7 days), there are no issues with monitoring speed, and it can be cost competitive with conventional laboratories.

Some possible (non-limiting) applications for the disclosed system and method include: CNS probe studies in neurosurgery, fraction collection in ICU monitoring, early detection of rejection episodes post transplant, glucose control using glucose oxidase, and pharmacokinetic research studies following the application of new medicaments.

Microdialysis is the method of choice for smaller molecules (Up to the size of 2000 molecular wt.). Complete equilibration goes with slower flow rates, so there is a limit to how low you can go with flow in a microdialysis system (0.5 ul/min). Regardless of flow, the size of the molecule is limited according to the catheter used. Thus, direct extraction of interstitial fluid has merit for some situations and can be used to measure larger molecules ex vivo.

Specifications: CNS microdialysis catheters: Hollow Fiber Specifications: 2 MWCO's: 13 and 18 kD; Working Volume: 5 μl/fiber; Fiber OD: 216 μm Fiber ID: 200 μm; Surface Area/Length: 6.3 mm2/cm; Volume/Length:0.31 μl/cm; **Q-Factor: 20; Fused Silica dimension:=0.0029″×0.0059″; *Q-factor is the ratio between membrane surface area and volume. The Q-factor is preferably as high as possible for maximum efficiency and to reduce dialysis time.

The present discourse broadly provides a system and method that is an improved point of care laboratory that is arranged to sense one or more chemical parameters within a mammalian body, and to transmit such parameters to an extra-corporeal receiver. In use, the device and receiver perform the method of continuously determining the chemical concentrations within a tract of a mammal. Previous embodiments of point of care laboratory devices have not been able to use continuous sensing because chemical means of capture has not been applied to this sensor. Antigen-Antibody capture sensing methods are not capable of measuring continuously, but the present technology is capable of measuring continuously for at least 10 days with no change of detection reagents. In some embodiments, this method includes the steps of implanting a microdialysis catheter membrane as a means of continuous body fluid sample collection, connecting the outflow of the microdialysis catheter to a microfluidics means of continuously exposing the chemical sensors in the device to the body fluids, receiving a signal that is proportional in strength to the concentration of each analyte in the body fluid, transmitting a signal from the point of care device, receiving the transmitted signal, and determining the real-time concentrations of substances in the body fluid of the mammal as a function of the received signal. The received signal also indicates the health of the mammal from interpretation of the measurements of one or more sensed parameters.

In some aspects a point of care laboratory device for use in continuous measurement of concentrations of substances in the body fluids extracted from an animal, comprising, an electric power source, a microdialysis catheter as a means of continuously sampling small amounts of body fluid from an animal, a means of continuously exposing the chemical detection apparatus to the body fluids as they are collected, a detection capability suitable for measurement of luminescence intensity, a radio signal transmitting means in enabling circuitry with said power source suitable for transmitting a radio signal which contains concentration information from the sensing composite, a radio signal receiving means for control and status monitoring of the device externally all encased in an outer shell that is configured to operate on or near the body of the animal. The extraction method for body sampling may be airflow from breath. The extraction method for body sampling may be from body fluid sampling from an implanted microdialysis catheter. The extraction method for body fluid sampling may be an implanted microneedles device.

The electric power source may comprise a battery of primary or secondary type or a suitable external electrical power source, and the transmitting means emits a radio signal, detectable exterior to said point of care laboratory device, when enabled by said power source. The power source may be controlled by a “Smart Power Control Algorithm” employing numerous control functions of electronic sub-systems to maximize battery life. A radio frequency receiver may be employed to allow external control of all device functions including data storage, data collection parameters, data transmission and device status.

In some embodiments a non-volatile memory for data storage is employed to retain collected data for transmission on request. The transmitting and receiving means may provide 256 digitally coded RF channels allowing the use of multiple devices in close proximity. Furthermore, the transmissions both to and from the device may be digitally encrypted to prevent unauthorized control of the device, or unauthorized use of patient data.

The point of care laboratory device of the present disclosure may include firmware for the device with an automatic calibration mode based upon a sensor exposed to a reference solution in a sealed chamber and a manual pre-use calibration mode based on active sensor exposure to reference solutions.

In some embodiments the point of care laboratory device of present disclosure uses a chemical sensing mechanism that functions continuously to detect chemicals in the fluids of the body for the entire time it is present on the body. The chemical sensing mechanism may use luminescence detection of analytes. In one embodiment, the present disclosure provides a device wherein the electromagnetic radiation generator provides a substrate for chemical sensors, and wherein the spectroscopic properties of the chemical sensors are modified upon contacting an analyte. In some embodiments, a method for the selective and simultaneous detection and quantification of multiple analytes is provided. In some embodiments, a method of making the device useful on fluids collected from the body of an animal on a continuous basis is provided.

In some embodiments, the point of care laboratory device of the present disclosure has one or more chemical sensors for interacting selectively with a particular analyte in a body fluid sample. In the absence of the analyte, the chemical sensor displays certain baseline spectroscopic properties characteristic of the sensor. However, when the analyte is present in the sample, the spectroscopic properties of the chemical sensor are modified. Detection and quantification of the analyte are based on a comparison of the modified properties and the baseline properties and the use of standard calibration methods that are well known to those skilled in the art of analytical chemistry.

In some embodiments, the point of care laboratory device uses a chemical sensor that comprises a reporter molecule whose optical properties are modified in the presence of an analyte. The properties of the sensor element may be directly modified upon its interaction with the analyte. The reporter molecule may be attached to a template material having a specific affinity for the analyte; as such, the optical properties of the reporter molecule are modified upon the interaction of the template material with the analyte. Thus, by the term “spectroscopic properties of the chemical sensor” or “chemical sensor's spectroscopic properties” it is meant the spectroscopic properties of the reporter molecule and vice versa. These properties may be optical in nature when the emitted electromagnetic radiation is within the visible spectrum i.e., between about 400 nm to about 800 nm. As an example if the chemical sensor is a site selectively templated and tagged xerogel (SSTTX) or a protein imprinted xerogel with integrated emission site (PIXIES), the reporter molecule is one or more luminescent reporter molecules within a molecularly templated xerogel and the analyte affinity is afforded by the template sites within the xerogel. In another example, where the chemical sensor is a luminescent ruthenium dye (tris (4,7-diphenyl-1, 10-phenanthroline) ruthenium (II), ([Ru (dpp) 3]2+), the reporter molecule ([Ru (dpp) 3]2+) provides an analyte-dependent response directly.

The point of care laboratory device can detect various types of analytes that may include both liquid and gaseous materials. These include CO2, O2, cytokines, interleukins, incretins, carbohydrates, hormones, hemoglobin, proteins, peptides, pesticides, drugs, herbicides, anions, cations, antigens, oligonucleotides, and haptens. Further, an embodiment the present disclosed system and method may indicate the pH and salinity of a sample. In addition, chemical sensors are available and may be used in the present disclosure to detect the presence of organic molecules such as polycyclic aromatic hydrocarbons, glucose, cholesterol, amino acids, and peptides. Further, the present disclosure provides a system and method to detect the presence of bacteria yeast, fungi and viruses of both normal and pathogenic nature. There are many more substances which can be detected, and the foregoing list is not to be considered exhaustive, but instead is merely representative.

The electromagnetic radiation emitted by the chemical sensor may be detected by any suitable method known in the art. A general configuration shows a detecting device in combination with a receiving and interpreting system. The receiving and interpreting system may have a receiver to receive electromagnetic radiation transmitted or emitted by the chemical sensor (s) and convert the optical signal into an electrical signal and an interpreter to interpret the received electrical signal. In one embodiment, the receiver is a complementary metal oxide semiconductor (CMOS) based array with a filter preceding the receiving surface on the CMOS array. The receiver may have a camera for recording images. The interpreter includes a controller and a computer having software running thereon. The receiving surface is connected to the controller.

One or more filters may be placed between the substrate and the receiving surface. The filter selectively passes desired wavelengths of the electromagnetic radiation moving from the detecting device toward the receiving surface and blocks undesired wavelengths. An example of a filter which can be used to practice the present disclosure is model number XF 3000-38 manufactured by Omega Optical of Brattleboro, Vt. This particular filter passes electromagnetic radiation above approximately 515 nm and strongly attenuates electromagnetic radiation below approximately 515 nm. Other filters or filter combinations are possible depending on the generator wavelength and the particulars associated with a given sensor. In some embodiments, the sample to be analyzed is continuously exposed to the chemical sensor(s), and the receiver components are placed in the proper position to permit the receiving and interpreting system to receive radiation from the chemical sensors. The electromagnetic information collected during operation can be digitized to provide input to a digital memory during sensing, and sent to a receiving device over wireless communications at time intervals. One or more SSTTX- or PIXIES-based sensors can be used. The luminescence output from these types of sensors is stable for many days under constant excitation. Thus, this demonstrates that using a method of the present disclosure, the chemical sensor platform is sufficiently stable to be used for detection and quantification of analytes in the body.

A point of care laboratory device according to an embodiment of the present disclosure uses the chemical sensing and analysis methods and systems contained in U.S. Pat. Nos. 6,241,948 and 6,589,438, which are incorporated by reference. Those methods and systems may be applied to a continuous body fluid measurement device as disclosed herein. The present disclosure provides a detecting device wherein the chemical sensor can be placed in contact with the electromagnetic radiation generator that excites the luminescent reporter molecules within the sensors, making the device compact and suitable for incorporation in the point of care laboratory device. Furthermore, the electromagnetic radiation used in the present disclosure is not reflected, filtered, or transmitted over a long distance prior to reaching the chemical sensors. The detecting device according to the present disclosure can be made relatively inexpensively and readily mass produced.

Xerogel-based sensor platform can be used to continuously detect one or more analyte molecules in body fluids in relation to concentration, wherein the luminescent signal emitted by the sensors occurs in strength proportional to analyte concentration in body fluids. The xerogel-based sensor platform associates and dissociates reversibly to its analyte molecule, enabling continuous signal emulation in proportion to changing concentration of said analyte molecules in body fluids. The molecular analysis substance can be any other composite or molecule which binds reversibly to its analyte molecule, enabling continuous luminescence signal emulation in relation to changing concentration of said analytes molecules in body fluids.

Some embodiments of the point of care laboratory device are capable of repeated detection of the concentration of at least one analyte in a sample and comprises: an electromagnetic radiation generating source having at least one SSTTX-or PIXIES-based sensor formed directly on or on a substrate that is in turn in close proximity to the electromagnetic radiation generating source such that the analyte containing stream can come into contact with the sensor, wherein the spectroscopic properties of the chemical sensor are modified in the presence of the analyte. In an embodiment, the electromagnetic radiation generating source is a light emitting diode. The system can further comprise a receiving and interpreting system having electromagnetic radiation receiver to receive electromagnetic radiation emitted by the chemical sensors, and having a capability to interpret the received electromagnetic radiation. The receiver can include a filter for selectively passing electromagnetic radiation. The receiver can also include a complementary metal oxide semiconductor (CMOS) based array a charge coupled device, and/or: a lens for focusing the electromagnetic radiation on the charge coupled device; an opaque shield above the lens for focusing the electromagnetic radiation on a complementary metal oxide semiconductor (CMOS) based array device. The sensing system can include: an interpreter with a means of storage for digitized data output from the complementary metal oxide semiconductor (CMOS) array.

The sensing system may further comprise a holding substrate for holding the chemical sensors in optical alignment with the electromagnetic radiation generating source, one or more filters, and a receiver. The holding substrate can be a xerogel or other material (e.g., glass, plastic) that is not degraded by the fluids of the body. The holding material can be comprised of tetramethylorthosilane.

In another embodiment the chemical sensor(s) is (are) comprised of a reporter molecule and an analyte-responsive template (e.g., SSTTX or PIXIES) having a specific affinity for the analyte. The reporter molecule can be selected from the group including fluorophore, phosphore and chromophore.

The body substance sampled continuously may include any of the following: the breath of the animal, blood, cerebrospinal fluid, fluid drainage from a wound or wound perfusion device, urine, vaginal fluid, tears, saliva, and/or gastrointestinal fluid.

The point of care laboratory device may include a fluid storage reservoir positioned exterior to the point of care laboratory device. The results of continuous analyte detection may trigger the analysis of the contents of the fluid storage reservoir. The body fluid from the storage reservoir may be recovered for laboratory measurement, PCR assay, and any other measurement. The body fluid from said storage reservoir may be recovered for a time averaged assay of analytes listed above upon collection.

The point of care laboratory device may provide chemical sensing on a continuous basis in any fluid or air containing environment of animals. The enabling circuitry of the point of care laboratory device may comprise a switching means and the quantified DC voltage signal can be digitized to provide input to a computer. Time multiplexed output of the multiple sensors may be converted to an intermediate frequency signal, quantified as a DC voltage signal and digitized to provide input to a computer. Transmitted signals may be received exterior of the animal body digitized and provided to a computer. The digitized information may have parameters comprising one for rate of progress of said monitored substance parameter in the patient or specific diseases of the patient. A computer may be programmed to scan and compute variations from pre-programmed factors. The computer may be programmed to initiate an actuating signal to the receiver than sampling of the reservoir should be carried out by the operator. The process and system disclosed herein involving initiation of the actuating signal may be operator controlled.

Capture of body fluid samples may initiate an indicator signal, by a capture indicator signal means comprised in the point of care laboratory device, which is detectable exterior of the body of the animal. Two or more point of care laboratory devices measuring two or more body fluids and transmitting differential signals may be utilized simultaneously.

In some embodiments the disclosure provides a process for the continuous collection of sensing data from the body of an animal and capture of body fluids for remotely triggered collection of sensing data comprising, a means of collecting body fluids, a point of care laboratory device containing a radio signal transmitting means suitable for determining concentrations of multiple analytes, transmitting a radio signal from the transmitting means; receiving the transmitted signal exterior of the body of the animal; digitizing the signal received; storing the digitized signal in computer recoverable, time sequence memory.

In some embodiments the disclosure provides a point of care laboratory device for continuous collection of sensing data from the body of an animal comprising, a non-digestible outer shell; an electric power source; a means of continuously collecting body fluids, a means of measuring the concentrations of one or more analytes on a continuous basis, a radio signal transmitting means in enabling circuitry with said power source suitable for transmitting a radio signal the strength of which determines the concentration of an analyte; a body fluid capture reservoir compartment, designed to be assayed in response to generation of said actuator signal. This embodiment may further include a means to receive a radio signal, and also a means to measure sensing signals from body fluids, and said output is converted to time multiplexed output.

The sensing means can be easily physically separated from the electronics including the power source, the data memory and the transmitting and receiving means. This provides the ability to replace/recharge the battery and store the collected data via a standard computer hardware communication interface.

The disclosed system and method may replace the laboratory testing that is carried out all over the world in every patient. Rather than getting a single test, it will be possible to test over 72 hours as often as desired, for a one time cost similar to the cost of one test. For countries without access to laboratories, this technology allows a full array of laboratory testing in home or doctors offices. The sensor device has many uses beyond medical, it can be used in food screening for toxins for example. The sensor device can be used to control pharmaceutical production by monitoring the quality of the synthesis processes used to manufacture drugs and biological. The disclosed system and method may also be used in food or wine production, where control of complex processes requires up to the minute information on changes in the mixture.

This device may replace laboratory testing, while one alternative is to keep using the laboratory testing that is familiar to both patients and physicians. This device was invented to add many lab tests to a profile that are not usually available in typical labs. By so doing, the reach of laboratory testing is extended to new markets. Finally, existing technology only works when a blood sample is drawn and sent to the lab. The present device works all the time, opening up a new more information rich laboratory environment to the physician. This method and device opens up large areas of the laboratory diagnostic field to exploration and treatment advantage.

This allows chemical sensing of multiple analytes at the bedside of the patient, the so-called “Point of Care.” The cost per test may be one tenth of the cost of the same test being sent to a laboratory. The hospital environment may require stability of the sensor over at least 3 days with minimal needs for re-calibration. The main requirement for the device is the need for continuous readout of changing conditions. Neither of these requirements can be achieved, without changing reagents, using any previously existing sensing technology.

Although the present invention has been described with respect to one or more particular embodiments, it will be understood that other embodiments of the present invention may be made without departing from the spirit and scope of the present invention. Hence, the present invention is deemed limited only by the appended claims and the reasonable interpretation thereof.

What is claimed is:

1. A device for continuous monitoring of a chemical parameter of an individual, comprising:
a microdialysis catheter having an inlet for receiving a perfusion fluid and an outlet for exiting the perfusion fluid containing dialysate;
a pump in fluid communication with the inlet of the catheter and configured to pump perfusion fluid through the microdialysis catheter; and
a flow-though monitor in fluid communication with the outlet of the catheter, wherein the monitor is configured to analyze the dialysate present in the perfusion fluid and detect the chemical parameter.

2. The device of claim 1, further comprising a reservoir in fluid communication with the monitor.

3. The device of claim 1, further comprising a transmitter configured to transmit monitored data from the monitor to a receiver.

4. The device of claim 1, wherein the flow-through monitor is removably attached to the individual.

5. A method for continuously monitoring a chemical parameter of an individual, comprising the steps of:
providing a continuous monitor including:
a microdialysis catheter having an inlet for receiving a perfusion fluid and an outlet for exiting the perfusion fluid containing dialysate;
a pump in fluid communication with the inlet of the catheter and configured to pump perfusion fluid through the microdialysis catheter; and
a flow-though monitor in fluid communication with the outlet of the catheter;
using the pump to cause perfusion fluid to flow into the inlet of the microdialysis cathether and to cause perfusion fluid and dialysate to exit the outlet of the microdialysis catheter, the perfusion fluid and dialysate flowing into the flow-through monitor; and
using the flow-through monitor to analyze the dialysate present in the perfusion fluid and detect the chemical parameter.
